(19) United States
(12) Patent Application Publication
(10) Pub. No.: US 2011/0250149 A1
(43) Pub. Date: Oct. 13, 2011

(54) Tiotropium Bromide Having a Low Degree of Crystallinity

(75) Inventors: Rajendra Narayanrao Kankan, Mumbai (IN); Dharmaraj Ramachandra Rao, Mumbai (IN); Maruti Ghagare, Thane (W) (IN)

(73) Assignee: CIPLA LIMITED, Mumbai (IN)

(21) Appl. No.: 13/127,348

(22) PCT Filed: Oct. 29, 2009
(86) PCT No.: PCT/GB2009/002575

§ 371 Date: Jun. 8, 2011

(30) Foreign Application Priority Data
Nov. 4, 2008 (IN) 2351/MUM/2008

(51) Int. Cl. A61K 31/79; A61K 9/12; C08F 8/34; C07D 491/18

(52) U.S. Cl. 424/43; 546/91; 424/78.24; 525/326.9

(57) ABSTRACT

The present invention provides tiotropium bromide having a low degree of crystallinity. The present invention also provides a complex of tiotropium bromide and polyvinylpyrrolidone, processes for preparing it and pharmaceutical formulations including it.

### CROSS-REFERENCE TO RELATED APPLICATIONS

This application is a filing under 35 U.S.C. 371 of International Application No. PCT/GB2009/002575 filed Oct. 29, 2009, entitled “Tiotropium Bromide Having a Low Degree of Crystallinity,” claiming priority of Indian Patent Application No. 2351/MUM/2008 filed Nov. 4, 2008, which applications are incorporated by reference herein in their entirety.

### FIELD OF INVENTION

The present invention relates to tiotropium bromide having a low degree of crystallinity, a process for preparing it and pharmaceutical compositions of it.

### BACKGROUND OF THE INVENTION

Tiotropium bromide was first disclosed in EP 0418716. Tiotropium bromide is a long-acting, anticholinergic bronchodilator used in the management of chronic obstructive pulmonary disease. On topical application it acts mainly on M₃ muscarinic receptors located in the airways to produce smooth muscle relaxation, thus producing a bronchodilatory effect. Tiotropium bromide capsules for inhalation are co-marketed by Boehringer-Ingelheim and Pfizer.

EP 1326862 and EP 1401445 describe monohydrate and anhydrous forms of tiotropium bromide, respectively.

WO 2007/075858 discloses the amorphous form of tiotropium bromide.

WO 2008/152398 describes a formulation comprising tiotropium bromide coated with polyvinylpyrrolidone (PVP). The coating requires multiple processing and may cause difficulties in scale-up due to overspray and lack of uniformity.

Pulmonary disorders are best treated when tiotropium bromide is administered in the form of inhalation. Suitable inhalation devices include metered dose inhalers (“MDIs”), dry powder inhalers and nebulizers. The manufacture of the abovementioned preparations depend upon various parameters related to the nature of the active substance. The actives can be either in crystalline or amorphous form.

The crystalline form of a drug compound may have advantages over an amorphous form. For example, a crystalline form may be more stable than an amorphous form, both before and during formulation and during subsequent storage. The bioavailability of a drug substance is affected by the physical properties of a drug substance such as by crystallinity, particle size, hygroscopicity, bulk density, flow characteristic, etc. Crystallization is a convenient method for purification of a drug substance on a larger scale, than other known techniques of purification such as chromatography. Also, when formulating a crystalline drug substance for delivery by inhalation, it is generally easier to mill or micronise a crystalline form to a respirable size (generally considered as particles less than 5 microns in diameter).

Crystalline forms are generally considered to be more stable but to tend to dissolve with slight difficulty; compared to the amorphous form which may have higher solubility but is generally less stable as it can convert to the crystalline form. Due to the absence of an ordered crystal lattice, the amorphous form requires minimal energy and thus provides the maximal solubility advantage as compared to the crystalline and hydrated forms of a drug. The apparent solubility and dissolution advantage offered by these systems is a vital approach to enhance the bioavailability of drugs. However, the limitations of amorphous systems such as physical instability and higher chemical reactivity, act as a hurdle in their extensive commercialization.

Therefore, it is important to understand the molecular and thermodynamic properties that contribute to the solubility and stability of drugs, and hence there is a need to develop a drug substance with physical properties that are suitable for pharmaceutical use.

### OBJECTS OF THE INVENTION

An object of the present invention is to provide tiotropium bromide having a low degree of crystallinity.

Another object of the present invention is to provide a tiotropium bromide-polyvinylpyrrolidone (PVP) complex.

Yet another object of the present invention is to provide a simple process for the preparation of tiotropium bromide having a low degree of crystallinity.

Still another object of the present invention is to provide tiotropium bromide with a high degree of stability and dose uniformity.

A further object of the present invention is to prepare a pharmaceutical composition containing tiotropium bromide having a low degree of crystallinity.

### SUMMARY OF THE INVENTION

According to a first aspect of the present invention, there is provided a complex of tiotropium bromide and polyvinylpyrrolidone (PVP). The PVP may be selected from the group consisting of PVP-K-12, PVP-K-15, PVP-K-17, PVP-K-25, PVP-K-30, PVP-K-60 and PVP-K-90. Suitably, the PVP may be PVP-K-25. The PVP may have a molecular weight ranging from 2500 to 1,200,000.

Advantageously, the tiotropium bromide has a degree of crystallinity lower than or equal to 75%, preferably lower than 70%.

According to another aspect of the present invention, there is provided tiotropium bromide having a degree of crystallinity lower than or equal to 75%, preferably lower than or equal to 70%. The degree of crystallinity may be below 75% and greater than 0%. Preferably, the degree of crystallinity is below 70% and greater than 0%. Suitably, the degree of crystallinity ranges from 10% to 75%, more preferably from 10% to 70%, still more preferably from 30% to 60%. The percent degree of crystallinity is determined as described below in the detailed description of the invention.

According to another aspect of the present invention, there is provided a process for preparing a tiotropium bromide-PVP complex, the process comprising preparing a solution of tiotropium bromide, PVP and a solvent, and isolating the tiotropium bromide-PVP complex from the solution. In an embodiment, the isolation comprises concentrating the solution under vacuum to obtain a residue, and drying the residue to obtain the tiotropium bromide-PVP complex.

In an embodiment, the solvent is selected from the group consisting of acetonitrile, methanol, water, dimethyl formamide, acetone, tetrahydrofuran, dimethyl sulfoxide and mixtures thereof, preferably acetone.

In an embodiment, the amount of PVP ranges from about 0.2% to about 90% by weight of the tiotropium bromide, preferably from about 10% to about 85% by weight of the tiotropium bromide, more preferably from about 20% to about 80% by weight of the tiotropium bromide, still more preferably from about 30% to about 70% by weight of the tiotropium bromide and yet more preferably from about 40% to about 60% by weight of the tiotropium bromide. Typically, the amount of PVP in the complex is about 50% by weight of the tiotropium bromide.

In an embodiment, the tiotropium bromide is mixed with the solvent to form a reaction mixture. The reaction mixture may be heated to a suitable temperature to obtain a clear solution. PVP may then be added to the reaction mixture. The solution may be concentrated under vacuum to obtain a residue. The residue may be washed with the same solvent as was used to form the solution and dried at a suitable temperature, preferably at a temperature ranging from about 30° C. to about 60° C., more preferably around 50° C., to obtain the tiotropium bromide-PVP complex.

According to another aspect of the present invention, there is provided a process for preparing a tiotropium bromide-PVP complex, the process comprising preparing a solution of tiotropium bromide, PVP and a solvent, and flash-evaporating the solvent. The solvent may be selected from the group consisting of a C1-C4 alcohol (for example, methanol, ethanol, i-propanol or butanol), acetone, water, acetonitrile, dichloromethane, or mixtures thereof.

In an embodiment, the solvent is flash-evaporated by applying heat and vacuum.

In another embodiment, the flash-evaporation is carried out by spray-drying.

According to another aspect of the present invention, there is provided a process for preparing a tiotropium bromide-PVP complex, the process comprising preparing a solution of tiotropium bromide, PVP and a solvent, freeze-drying the solution and lyophilizing the freeze-dried solution. The solvent may be selected from the group consisting of water, a C1-C4 alcohol (for example, methanol, ethanol, i-propanol or butanol), acetone and tetrahydrofuran (THF).

The tiotropium bromide used in the processes of the present invention may be in any crystalline form. The tiotropium bromide used in the process of the present invention may be a hydrate form, anhydrous, a derivative thereof, or in prodrug form.

In an embodiment, the tiotropium bromide-PVP complex described above has been prepared by any one of the processes described above.

According to another aspect of the present invention, there is provided a pharmaceutical composition comprising a tiotropium bromide-PVP complex as described above together with one or more pharmaceutically acceptable excipients.

In an embodiment, the formulation is suitable for administration by inhalation. Suitably, the formulation is an aerosol.

In an embodiment, the formulation comprises the tiotropium bromide-PVP complex, at least one hydrofluoroalkane propellant and optionally one or more pharmaceutically acceptable excipients. Suitably, the or each pharmaceutically acceptable excipient is a bulking agent and/or a co-solvent. The bulking agent may be lactose. he co-solvent may be polyethylene glycol (PEG). In an embodiment, the formulation consists of the tiotropium bromide-PVP complex, lactose, and a hydrofluoroalkane such as HFA 227 (1,1,1,2,3,3,3-heptafluoropropane).

### BRIEF DESCRIPTION OF THE DRAWINGS

FIG. 1 shows an XRPD of a tiotropium bromide-PVP complex of the present invention.

### DETAILED DESCRIPTION OF THE INVENTION

Drugs like tiotropium that show therapeutic efficacy at very low concentrations need to be dispensed in a manner such that they provide a high degree of homogeneity and only a slight fluctuation in the dispersion characteristics.

Tiotropium bromide administered preferably by inhalation needs to fulfill certain parameters such as homogeneity of the powder mixture, reproducible release of the active in constant amounts, and low variability and stability under various environmental conditions, during production and in the final composition. These are essential requirements in order to prevent the use of compositions that contain breakdown products of the active.

The nature of tiotropium bromide in the formulation in turn has an effect on the parameters. The crystalline form is known to dissolve with difficulty while the amorphous form which, although it has a higher solubility, tends to get readily converted to the crystalline form.

Accordingly, the present invention provides a form of tiotropium bromide with a high degree of solubility and enhanced absolute bioavailability while also preserving the physical and chemical stability required for formulation.

The inventors of the present invention have also surprisingly found that, when used as a carrier for tiotropium bromide, polyvinylpyrrolidone (PVP) increases the stability and solubility of tiotropium bromide by decreasing the degree of crystallinity of the tiotropium bromide.

The tiotropium bromide-PVP complex of the present invention exists not as a mere physical mixture, but as a complex with its own unique physical/chemical properties.

PVP is a homopolymer of N-vinyl-2-pyrrolidone on which the tiotropium bromide gets adsorbed. Complexation of tiotropium bromide with PVP is a non-covalent stacking type of association between non-polar areas of tiotropium bromide and PVP. The complex shows no agglomeration of the drug particles as compared to the crystalline form and has better stability than the amorphous form. The complex shows good aerosol dispersion and higher solubility for inhalation drug delivery.

The tiotropium bromide complex of the present invention has unique physical and chemical properties, which properties are significantly different from those of a physical mixture of tiotropium bromide and PVP. A physical mixture of tiotropium bromide tends to form an agglomeration of the drug substance and PVP. The physical mixture does not have any synergistic effect whereas the complex of the present invention has a significant effect on the properties and stability of the drug substance thereby affecting its bioavailability and efficacy which effect is not seen in a physical mixture.

The present invention thus provides tiotropium bromide having a low degree of crystallinity which is more suitable for pharmaceutical preparations as compared to pure crystalline and amorphous forms.

In another aspect, the present invention provides tiotropium bromide having a low degree of crystallinity that may be obtained by complexing with polyvinylpyrrolidone (PVP).

Polyvinylpyrrolidones (PVP) are cross-linked polymers classified either on the basis of K-value as PVP-K-12, PVP-K-15, PVP-K-17, PVP-K-25, PVP-K-30, PVP-K-60, PVP-K-90 or molecular weight as ranging from PVP-2500 to 1,200,000.

In an embodiment, the present invention provides a method for preparing tiotropium bromide having a low degree of crystallinity. The method comprises mixing tiotropium bromide with a suitable solvent. The reaction mixture may be heated to a suitable temperature to obtain a clear solution. PVP is then added to the reaction mixture. This solution is concentrated under vacuum to obtain a residue. The residue is washed with the same solvent and dried at a suitable temperature preferably 50° C. to obtain a tiotropium bromide-PVP complex.

The solvent used in the above process may be selected from acetonitrile, methanol, water, dimethyl formamide, acetone, tetrahydrofuran or dimethyl sulfoxide, preferably acetone.

In an embodiment, the amount of PVP should be between about 0.2% and about 90% by weight of the tiotropium bromide, preferably between about 10% and about 80% by weight of the tiotropium bromide, more preferably, between about 20% and about 70% by weight of the tiotropium bromide, still more preferably between about 30% and about 60% by weight of the tiotropium bromide, most preferably around 50% by weight by weight of the tiotropium bromide.

Alternatively, tiotropium bromide having a low degree of crystallinity can be isolated by “flash-evaporating” the solvent. A flash-evaporating technique with respect to this invention means removal of the solvent by applying heat and vacuum. Preferably, the temperature in the heating step ranges from about 40° C. to about 70° C., preferably from about 45° C. to about 60° C., most preferably from about 50° C. to about 55° C.

In an embodiment, flash evaporation is carried out by spray-drying.

Alternatively, isolation of tiotropium bromide having a low degree of crystallinity can be carried out by lyophilization.

The tiotropium bromide used in the process of the present invention may be in any crystalline form. The tiotropium bromide used in the process of the present invention may be a hydrate form, anhydrous, a derivative thereof, or in prodrug form.

In another aspect, the present invention provides a pharmaceutical composition comprising a tiotropium bromide-PVP complex and one or more pharmaceutically acceptable carriers.

In an embodiment, the degree of crystallinity of the tiotropium bromide-PVP complex can be measured with X-ray powder diffraction (XRPD). For this analysis, a thin layer of the triturated sample is smeared onto cut silicon single crystal zero background holder. Cu KV radiation and constant or automatic anti scatter and divergence slits are used to obtain a diffractogram with 2θ values from 3° to at least 40°.

The degree of crystallinity is calculated with the formula:

% Crystallinity=100A/(A+B−C)

A=total area of peak arising from diffraction from the crystalline fraction of the sample (i.e., crystalline region)
B=total area below area A (i.e., amorphous and background region)
C=background area (due to air scattering, fluorescence, equipment, etc)

Area calculations are performed for a 2θ range of 3°-40°.

Background area (arising only from the instrument parameters) is subtracted using manual spline, area of this profile from 3°-40° 2θ is calculated which gives the area for amorphous and crystalline areas of the sample, i.e., (A+B−C).

The XRD pattern is subjected to normal background subtraction that eliminates background area arising due to both amorphous and instrumental parameter. This area is taken as the crystalline area of the pattern (A).

In an embodiment, there is provided tiotropium bromide having a degree of crystallinity which is below 75%. More preferably, the degree of crystallinity is below 70%. The degree of crystallinity may be below 75% and greater than 0%. Preferably, the degree of crystallinity is below 70% and greater than 0%. Suitably, the degree of crystallinity ranges from 10% to 75%, more preferably from 10% to 70%, still more preferably from 30% to 60%. The percent degree of crystallinity is determined as described above.

### EXAMPLES

There follow, by way of non-restrictive explanation of the present invention, the following examples.

### Example 1

### TTB-PVP Complex

5 g of tiotropium bromide was introduced into a reaction vessel. Acetone was added. The reaction mixture was heated to a temperature of 50-55° C. Water (15 ml) was added to the reaction mixture to obtain a clear solution. To this solution, 2.5 g of PVP-K-25 was added. The solution was concentrated under vacuum to obtain a residue. The residue was washed with acetone (15 ml) and dried under vacuum at 50° C. to obtain the title complex (6 g).

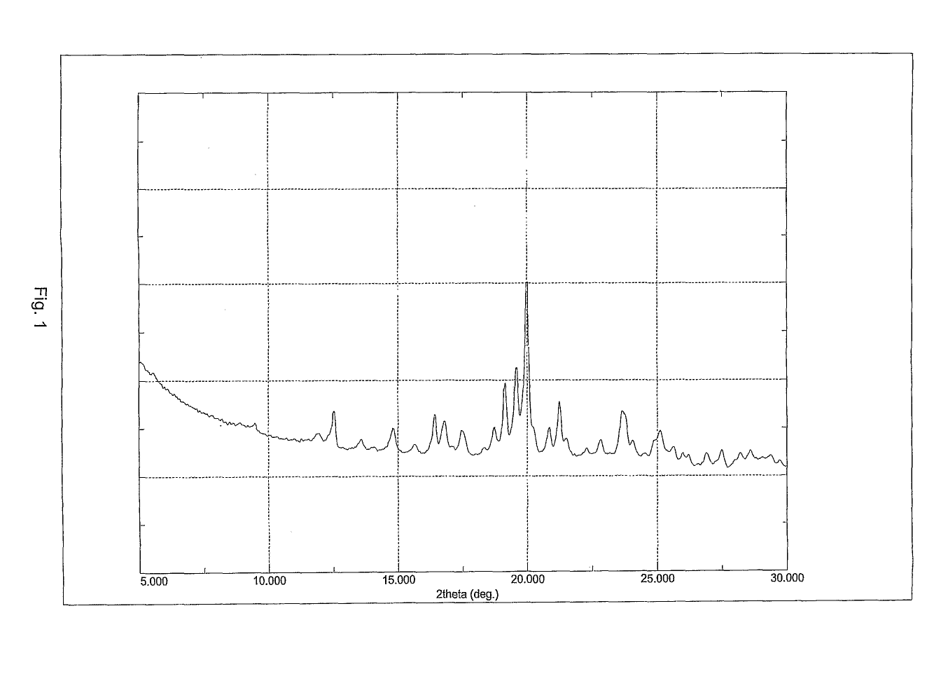

The XRPD of the Example 1 complex is shown in FIG. 1. From this XRPD, and the formula quoted above, the degree of crystallinity was calculated as being 58.35%.

### Example 2

### TTB-PVP Complex

5 g of tiotropium bromide monohydrate was introduced into a reaction vessel. Water (15 ml) was added to the reaction mixture to obtain a clear solution. To this solution, 2.5 g of PVP-K-17 was added. The solution was concentrated under vacuum to obtain a residue. The residue was washed with acetone (15 ml) and dried under vacuum at 50° C. to obtain the title complex (6 g).

### Example 3

### TTB-PVP Complex

5 g of tiotropium bromide monohydrate was introduced into a reaction vessel. Acetone was added. The reaction mixture was heated to a temperature of 50-55° C. Water (15 ml) was added to the reaction mixture to obtain a clear solution. To this solution, 0.25 g of PVP-K-25 was added. The solution was concentrated under vacuum to obtain a residue. The residue was washed with acetone (15 ml) and dried under vacuum at 50° C. to obtain the title complex (5.2 g) (% crystallinity−67.42%).

### Example 4

### TTB-PVP Complex Formed by Lyophilization

5 g of tiotropium bromide and 2.5 g of polyvinylpyrrolidone (PVP) were stirred in 50 ml of water at 25-30° C. until dissolved. The solution was quick-frozen in a dry ice bath. The solution was lyophilized at a condenser temperature of approximately 0° C. in the presence of a high vacuum. The resultant title complex was obtained in the form of a solid (5.8 g).

### Example 5

### TTB-PVP-K-25 Complex Formed by Evaporation

2.5 g of tiotropium bromide and 1.25 g of polyvinylpyrrolidone (PVP) were stirred in 10 ml of ethanol at 25-30° C. until dissolved. The solution was poured into a polyethylene tray and the ethanol evaporated in a vacuum oven in the presence of a nitrogen stream. The resultant dry solid title product was isolated (2.5 g).

### Example 6

### TTB-PVP-K-17 Complex Formed by Spray Drying

2.5 g of tiotropium bromide was dissolved in 25 ml of methanol. 1.25 g of PVP was dissolved in methanol (15 ml). The two solutions were mixed and spray dried. The following parameters were used:

Instrument—Labultima LU-222 ADVANCE SPRAY DRYER

Temperature—50-55° C.

Vacuum—40 mmWC

N₂ pressure—1-2 kg

Feeding rate—3 ml/min.

The solid product was collected in a collector (2.0 g).

### Example 7

### Pharmaceutical Formulation of Tiotropium Bromide-PVP Complex

Tiotropium bromide—PVP complex 1.8 mg

Lactose 1.8 mg

HFA¹—227 Q.S.

¹HFA=1,1,1,2,3,3,3-heptafluoropropane

Lactose was added to a canister containing the tiotropium bromide-PVP complex. The canister was crimped with the metered valve and the propellant charged in the canister.

It will be appreciated that the invention may be modified within the scope of the appended claims.

1. A complex of tiotropium bromide and polyvinylpyrrolidone (PVP).

2. The complex according to claim 1, wherein the PVP is selected from the group consisting of PVP-K-12, PVP-K-15, PVP-K-17, PVP-K-25, PVP-K-30, PVP-K-60 and PVP-K-90.

3. The complex according to claim 1, wherein the PVP has a molecular weight ranging from 2500 to 1,200,000.

4. The complex according to claim 1, wherein the tiotropium bromide has a degree of crystallinity lower than or equal to 75%.

5. The complex according to claim 4, wherein the tiotropium bromide has a degree of crystallinity lower than or equal to 70%.

6. Tiotropium bromide having a degree of crystallinity lower than or equal to 75% and greater than 0%.

7. Tiotropium bromide according to claim 6, having a degree of crystallinity lower than or equal to 70%.

8. A process for preparing a tiotropium bromide-PVP complex, the process comprising preparing a solution of tiotropium bromide, PVP and a solvent, concentrating the solution under vacuum to obtain a residue, and drying the residue to obtain the tiotropium bromide-PVP complex.

9. The process according to claim 8, wherein the solvent is selected from the group consisting of acetonitrile, methanol, water, dimethyl formamide, acetone, tetrahydrofuran and dimethyl sulfoxide.

10. The process according to claim 8, wherein the amount of PVP ranges from about 0.2% to about 90% by weight of the tiotropium bromide.

11. The process according to claim 10, wherein the amount of PVP ranges from about 40% to about 60% by weight of the tiotropium bromide.

12. A process for preparing a tiotropium bromide-PVP complex, the process comprising preparing a solution of tiotropium bromide, PVP and a solvent, and flash-evaporating the solvent.

13. The process according to claim 12, wherein the solvent is flash-evaporated by applying heat and vacuum.

14. The process according to claim 12, wherein the flash-evaporation is carried out by spray-drying.

15. A process for preparing a tiotropium bromide-PVP complex, the process comprising preparing a solution of tiotropium bromide, PVP and a solvent, freeze-drying the solution and lyophilizing the freeze-dried solution.

16. The tiotropium bromide-PVP complex according to claim 1, prepared by a process comprising preparing a solution of tiotropium bromide, PVP and a solvent, concentrating the solution under vacuum to obtain a residue, and drying the residue to obtain the tiotropium bromide-PVP complex.

17. A pharmaceutical composition comprising a tiotropium bromide-PVP complex according to claim 1, together with one or more pharmaceutically acceptable excipients.

18. The pharmaceutical composition according to claim 17, wherein the formulation is suitable for administration by inhalation.

19. The pharmaceutical composition according to claim 17, wherein the formulation is an aerosol.

20. The pharmaceutical composition according to claim 17, wherein the formulation comprises a propellant.

21-23. (canceled)
